(19) United States
(12) Patent Application Publication
(10) Pub. No.: US 2003/0228308 A1
(43) Pub. Date: Dec. 11, 2003

(54) Isolated peptides which bind to HLA-Cw6 molecules and uses thereof

(76) Inventors: Yi Zhang, Brussels (BE); Catia Traversari, Milano (IT); Thierry Boon-Falleur, Brussels (BE); Pierre Van der Bruggen, Brussels (BE)

Correspondence Address: FULBRIGHT & JAWORSKI, LLP, 666 FIFTH AVE, NEW YORK, NY, 10103-3198, US

(21) Appl. No.: 10/164,121

(22) Filed: Jun. 5, 2002

(51) Int. Cl. C12Q 1/68; G01N 33/574; C07H 21/04; A61K 39/395; C07K 14/74; C12N 5/08

(52) U.S. Cl. 424/144.1; 435/6; 435/7.23; 435/372; 530/350; 536/23.2

(57) ABSTRACT

A peptide, previously identified as a binding partner of HLA-Cw3 and Cw16, has now been found to bind to HLA-Cw6. The therapeutic and diagnostic ramifications of this are the subject of this invention, as are various products obtained in the course of the development of the invention.

### FIELD OF THE INVENTION

This invention relates to peptides which form immunologically active complexes with MHC molecules. More particularly, it involves peptides based upon amino acid sequences found in the molecule referred to as “MAGE-1,” which form complexes with the MHC molecule HLA-Cw6.

### BACKGROUND AND PRIOR ART

The study of the recognition or lack of recognition of cancer cells by a host organism has proceeded in many different directions. Understanding of the field presumes some understanding of both basic immunology and oncology.

Early research on mouse tumors revealed that these displayed molecules which led to rejection of tumor cells when transplanted into syngeneic animals. These molecules are “recognized” by T cells in the recipient animal, and provoke a cytolytic T cell response with lysis of the transplanted cells. This evidence was first obtained with tumors induced in vitro by chemical carcinogens, such as methylcholanthrene. The antigens expressed by the tumors and which elicited the T cell response were found to be different for each tumor. See Prehn, et al., J. Natl. Canc. Inst. 18: 769-778 (1957); Klein, et al., Cancer Res. 20:1561-1572 (1960); Gross, Cancer Res. 3:326-333 (1943), Basombrio, Cancer Res. 30:2458-2462 (1970) for general teachings on inducing tumors with chemical carcinogens and differences in cell surface antigens. This class of antigens has come to be known as “tumor specific transplantation antigens” or “TSTAs”. Following the observation of the presentation of such antigens when induced by chemical carcinogens, similar results were obtained when tumors were induced in vitro via ultraviolet radiation. See Kripke, J. Natl. Canc. Inst. 53:333-1336 (1974).

While T cell mediated immune responses were observed for the types of tumor described supra, spontaneous tumors were thought to be generally non-immunogenic. These were therefore believed not to present antigens which provoked a response to the tumor carrying subject. See Hewitt, et al., Brit. J. Cancer 33:241-259 (1976).

The family of tum⁻ antigen presenting cell lines are immunogenic variants obtained by mutagenesis of mouse tumor cells or cell lines, as described by Boon, et al., J. Exp. Med. 152:1184-1193 (1980), the disclosure of which is incorporated by reference. To elaborate, tum⁻ antigens are obtained by mutating tumor cells which do not generate an immune response in syngeneic mice and will form tumors (i.e., “tum⁺” cells). When these tum⁺ cells are mutagenized, they are rejected by syngeneic mice, and fail to form tumors (thus “tum^{-”).} See Boon, et al., Proc. Natl. Acad. Sci USA 74:272 (1977), the disclosure of which is incorporated by reference. Many tumor types have been shown to exhibit this phenomenon. See, e.g., Frost, et al., Cancer Res. 43:125 (1983).

It appears that tum⁻ variants fail to form progressive tumors because they elicit an immune rejection process. The evidence in favor of this hypothesis includes the ability of “tum⁻” variants of tumors, i.e., those which do not normally form tumors, to do so in mice with immune systems suppressed by sublethal irradiation, Van Pel, et al. Proc. Natl, Acad. Sci. USA 76:5282-5285 (1979); and the observation that intraperitoneally injected tum⁻ cells of mastocytoma P815 multiply exponentially for 12-15 days, and then are eliminated in only a few days in the midst of an influx of lymphocytes and macrophages (Uyttenhove, et al., J. Exp. Med. 152:1175-1183 (1980)). Further evidence includes the observation that mice acquire an immune memory which permits them to resist subsequent challenge to the same tum⁻ variant, even when immunosuppressive amounts of radiation are administered with the following challenge to the same tum⁻ variant, even when immunosuppressive amounts of radiation are administered wit the following challenge of cells (Boon, et al., Proc. Natl, Acad. Sci. USA 74:272-275 (1977); Van Pel, et al., supra; Uyttenhove, et al., supra). Later research found that when spontaneous tumors were subjected to mutagenesis, immunogenic variants were produced which did generate a response. Indeed, these variants were able to elicit an immune protective response against the original tumor. See Van Pel, et al., J. Exp. Med. 157:1992-2001 (1983). Thus, it has been shown that it is possible to elicit presentation of a so-called “tumor rejection antigen” in a tumor which is a target for a syngeneic rejection response. Similar results have been obtained when foreign genes have been transfected into spontaneous tumors. See Fearon, et al., Cancer Res. 48:2975-1980 (1988) in this regard.

A class of antigens has been recognized which are presented on the surface of tumor cells and are recognized by cytotoxic T cells, leading to lysis. This class of antigens will be referred to as “tumor rejection antigens” or “TRAs” hereafter. TRAs may or may not elicit antibody responses. The extent to which these antigens have been studied, has been via cytolytic T cell characterization studies, in vitro i.e., the study of the identification of the antigen by a particular cytolytic T cell (“CTL” hereafter) subset. The subset proliferates upon recognition of the presented tumor rejection antigen, and the cells presenting the antigen are lysed. Characterization studies have identified CTL clones which specifically lyse cells expressing the antigens. Examples of this work may be found in Levy et al., Adv. Cancer Res. 24:1-59 (1977); Boon, et al., J. Exp. Med. 152:1184-1193 (1980); Brunner, et al., J. Immunol. 124:1627-1634 (1980); Maryanski, et al., Eur. J. Immunol. 124:1627-1634 (1980); Maryanski, et al., Eur. J. Immunol. 12:406-412 (1982); Palladino, et al., Canc. Res. 47:5074-5079 (1987). This type of analysis is required for other types of antigens recognized by CTLs, including minor histocompatibility antigens, the male specific H—Y antigens, and a class of antigens, referred to as “tum⁻” antigens, and discussed herein.

A tumor exemplary of the subject matter described supra is known as P815. See DePlaen, et al, Proc. Natl. Acad. Sci. USA 85:2274-2278 (1988); Sikora, et al., EMBO J 9:1041-1050 (1990), and Sibille, et al., J. Exp. Med. 172:35-45 (1990), the disclosures of which are incorporated by reference. The P815 tumor is a mastocytoma, induced in a DBA/2 mouse with methylcholanthrene and cultured as both an in vitro tumor and a cell line. The P815 line has generated many tum⁻ variants following mutagenesis, including variants referred to as P91A (DePlaen, supra), 35B (Szikora, supra) and P198 (Sibille, supra). In contrast to tumor rejection antigens—and this is a key distinction—the tum⁻ antigens are only present after the tumor cells are mutagenized. Tumor rejection antigens are present on cells of a given tumor without mutagenesis. Hence, with reference to the literature, a cell line can be tum⁺, such as the line referred to as “P1”, and can be provoked to produce tum⁻ variants. Since the tum⁻ phenotype differs from that of the parent cell line, one expects a difference in the DNA of tum⁻ cell lines as compared to their tum⁺ parental lines, and this difference can be exploited to locate the gene of interest in tum⁻ cells. As a result, it was found that genes of tum⁻ variants such as P91A, 35B and P198 differ from their normal alleles by point mutations in the coding regions of the gene. See Szikora and Sibille, supra, and Lurquin, et al., Cell 58:293-303 (1989). This has proved not to be the case with the TRAs of this invention. These papers also demonstrated that peptides derived from the tum⁻ antigen are presented by the L^{d} molecule for recognition by CTLs. P91A is presented by L^{d}, P35 by D^{d} and P198 by K^{d}.

U.S. Pat. No. 5,342,774, the disclosure of which is incorporated by reference, disclosed three members of a family of the genes referred to hereafter as the “MAGE” family of genes. MAGE-1, 2 and 3 are disclosed therein. Also see Traversari, et al., J. Exp. Med 176:1453-1457 (1993); Science 254:1643-147 (1991), the disclosures of which are incorporated by reference. Additional members of the MAGE family have been discovered and are disclosed in, e.g., DePlaen, et al., Immunogenetics 40:360 (1994), and U.S. Pat. No. 5,612,201 to DePlaen, both of which are incorporated by reference. With respect to MAGE-1, in addition to the '774 patent, see e.g. U.S. Pat. No. 5,925,729.

The genes are useful as a source for the isolated and purified tumor rejection antigen precursor and the TRA themselves, either of which can be used as an agent for treating the cancer for which the antigen is a “marker”, as well as in various diagnostic and surveillance approaches to oncology, discussed infra. It is known, for example that tum⁻ cells can be used to generate CTLs which lyse cells presenting different tum⁻ cells can be used to generate CTLs which lyse cells presenting different tum⁻ antigens as well as tum⁺ cells. See, e.g., Maryanski, et al., Eur. J. Immunol 12:401 (1982); and Van den Eynde, et al., Modern Trends in Leukemia IX (June 1990), the disclosures of which are incorporated by reference. The tumor rejection antigen precursor may be expressed in cells transfected by the gene, and then used to generate an immune response against a tumor of interest.

In the parallel case of human neoplasms, it has been observed that autologous mixed lymphocyte-tumor cell cultures (“MLTC” hereafter) frequently generate responder lymphocytes which lyse autologous tumor cells and do not lyse natural killer targets, autologous EBV-transformed B cells, or autologous fibroblasts (see Anichini, et al., Immuno. Today 8:385-389 (1987)). This response has been particularly well studied for melanomas, and MLTC have been carried out either with peripheral blood cells or with tumor infiltrating lymphocytes. Examples of the literature in this area including Knuth, et al., Proc. Natl. Acad. Sci. USA 86:2804-2802 (1984); Mukherji, et al., J Exp. Med. 158:240 (1983); Hérin, et al., Int. J. Canc. 39:390-396 (1987); Topalian, et al., J. Clin. Oncol 6:839-853 (1988). Stable cytotoxic T cell clones (“CTLs” hereafter) have been derived from MLTC responder cells, and these clones are specific for the tumor cells. See Mukherji, et al., supra, Hérin, et al., supra, Knuth, et al., supra. The antigens recognized on tumor cells by these autologous CTLs do not appear to represent a cultural artifact, since they are found on fresh tumor cells. Topalian, et al., supra; Degiovanni, et al., Eur. J. Immul. 20:1865-1868 (1990). These observations, coupled with the techniques used herein to isolate the genes for specific murine tumor rejection antigen precursors, have led to the isolation of nucleic acid sequences coding for tumor rejection antigen precursors of TRAs presented on human tumors. It is now possible to isolate the nucleic acid sequences which code for tumor rejection antigen precursors, including, but not being limited to those most characteristic of a particular tumor, with ramifications that are described infra.

Additional work has focused upon the presentation of TRAs by the class of molecules known as human leukocyte antigens, or “HLAs”. This work has resulted in several unexpected discoveries regarding the field. Specifically, in U.S. Pat. No. 5,405,940, the disclosure of which is incorporated by reference, nonapeptides including a MAGE-3 derived peptide, are taught which are presented by HLA-A1 molecules. The reference teaches that given the known specificity of particular peptides for particular HLA molecules, one should expect a particular peptide to bind one HLA molecule, but not others. This is important, because different individuals possess different HLA phenotypes. As a result, while identification of a particular peptide as being a partner for a specific HLA molecule has diagnostic and therapeutic ramifications, these are only relevant for individuals with that particular HLA phenotype. There is a need for further work in the area, because cellular abnormalities are not restricted to one particular HLA phenotype, and targeted therapy requires some knowledge of the phenotype of the abnormal cells at issue.

Additional peptides have been identified which consist of amino acid sequences found in MAGE-1, but which bind to different MHC molecules. See, e.g., U.S. Pat. Nos. 5,405,940 and 5,925,729 which describe peptides which bind to HLA-A1 molecules, and also see U.S. Pat. Nos. 5,558,995 and 6,228,971, which teach peptides consisting of amino acid sequences found in MAGE-1, which bind to HLA-Cw*1601 molecules. CTLs obtained from two melanoma patients after mixed lymphocyte-tumor cell cultures have been found to recognized MAGE-1 based peptides presented by HLA-A1, B37 and Cw16 molecules. See, e.g., Tanzarella, et al., Canc. Res. 59:2668-71 (1999); Traversari, et al., Immunogenetics 35:45-152 (1992); van der Bruggen, et al., Eur. J. Immunol. 24:2134-2140 (1994), all of which are incorporated by reference. Also see, e.g., Fujie, et al., Int. J. Cancer 80:169-172 (1999), who used synthetic peptides, based upon motif analysis such as that taught by Ramensee, supra, to develop synthetic peptides which should be good binding to HLA-A24, to develop relevant CTLs. Many other peptides have been identified which bind to different HLA molecules, including HLA-A3 (Chaux, et al., J. Immunol 163:2928-2836 (1999); A68 (Chaux, et al, ibid.) B7 (Luiten, et al., Tissue Antigens 55:149:152 (2000)), B35 (Luiten, et al., Tissue Antigens 56:77-81 (2000)); B53 (Chaux, et al, supra); Cw2 (Chaux, et al, supra); Cw3 (Chaux, et al, supra); DR13 (Chaux, et al, J. Exp. Med. 189:767-78 (1999); and DR15 (Chaux, et al., Eur. J. Immunol 31:1910-6 (2001)).

It is important to note that different approaches have been taken to identifying the peptides described herein with different ramifications. For example, Traversari, et al. and Tanzarella, et al., supra, secured CTLs from melanoma patients following autologous, mixed lymphocyte tumor cell cultures. Fujie, et al., using information found in, e.g., Ramensee, et al., Immunogenetics 41:178-228 (1995), incorporated by reference, was applied to the complete sequence of MAGE-1 protein to identify potential HLA molecule binders. These were then tested, and active molecules identified thereby.

This approach, i.e., employing motif analysis, has been found to exhibit a major drawback in that several peptide specific CTL generated using the synthetic peptides, do not recognize HLA matched tumor cells which express MAGE-1 endogenously. There have been two explanations proposed for this. One is that the peptides at issue are not generated efficiently by the cells. The second is that the CTLs obtained using high concentrations of the synthetic peptides have low affinity for the target. See Dahl, et al., J. Immunol 157:239-246 (1996).

MAGE-1 is expressed in a variety of cancers. Data indicate that it is expressed in 53% of esphoageal cancers, 49% of non-small cell lung carcinoma, and 48% of metastatic melanomas. See, e.g., Boon, et al., “Cancer Vaccines: Cancer Antigens, Shared Tumor Specific Antigens” in Rosenberg, ed., Principles and Practice of The Biologic Therapy of Cancer (Philadelphia, JB Lippincott Williams & Wilkins, 2000), pp. 493-504; Van den Eynde, et al., Cancer Immunity 2001: www.cancerimmunity.org/peptidedatabase/tcellepitopes.htm:; Van den Eynde, et al., Curr. Opin. Immunol 9:684-693 (1997).

A new strategy has been developed for identifying only will processed tumor antigens:dendritic cells transduced with gene MAGE-1 are used as stimulator cells for autologous CD8⁺ T cells. See, e.g., Luiten, et al., Tissue Antigens 55:149-152 (2000); Chaux, et al., J. Immunol 163:2928-36 (1999); Luiten, et al., Tissue Antigens 56:77-81 (2000); Schultz, et al., Tissue Antigens 57:103-9 (2001), and Van den Eynde: Cancer Immunity 2001: www.cancerimmunity.org/peptidedatabase/tcellepitopes.htm:, all of which are incorporated by reference, for examples of the application of this technique, with identification of relevant antigenic peptides.

Marsh, et al., The HLA Factsbook, (Academic Press, 2000), incorporated by reference, supplements older information on MHC binding peptides, such as that provided by Ramensee, supra. Relevant here is Marsh's discussion of the MHC molecule HLA-Cw6 which is not presented in Ramensee. Marsh, et al. note that approximately 15% of the black population, approximately 10% of caucasian and abariginal populations and about 7% of oriental and Amerindian populations present HLA-Cw6 alleles (four have been identified). With respect to binding peptides, rather than presenting a traditional anchor pattern of at least two well defined amino acids, one of which is at the C terminus, the only “common denominator” observed by Marsh, et al. is the amino acid “L” at position 9; however, of the two T cell epitopes disclosed by Marsh, et al., do not present this amino acid.

As will be seen herein, it has now been observed that a MAGE-1 peptide, previously identified as a T cell epitope for HLA-Cw3 and Cw16, is a T cell epitope for Cw6. This, and the ramifications of this observation, constitute the invention, which is elaborated upon in the detailed description which follows.

### EXAMPLE 1

This example describes experiments in which monocyte derived dendritic cells were infected with a canarypox virus that had been engineered to contain the MAGE-1 coding sequence. The construct, referred to as “ALVAC-1” hereafter, was obtained from a commercial source.

Peripheral blood cells were obtained from a hemochromatosis patient as standard, buffy coat preparations. These preps were laid down on a 15 ml Lymphoprep layer, in 50 ml tubes. Contamination of the PBMCs by platelets was minimized by centrifuging the samples for 20 minutes at room temperature at 1000 rpms, followed by removal of the top 20-25 mls, which contain most of the platelets. The tubes were then recentrifuged, at 1500 rpms for twenty minutes, at room temperature. The interphases, which contain the PBMCs, were harvested and washed at least 3 times in cold phosphate buffer solution containing 2 mM EDTA, to eliminate the remaining platelets.

Antologous dendritic cells were then generated by depleting PBMCs from T lymphocytes, via rosetting with sheep erythrocytes that had been treated with 2-aminoethyliso thiouronium. Rosetted T cells were then treated with NH₄Cl (160 mM), in order to lyse the sheep erythrocytes, and were then washed. The CD8⁺ T lymphocytes in the preps were isolated from the rosetted T cells by positive selection, using an anti CD8⁺ monoclonal antibody, coupled to magnetic microbeads. The resulting dendritic cells were sorted through a magnet, and frozen until needed.

To infect the resulting purified dendritic cells, 4×10⁶ cells were combined with ALVAC-MAGE-1, at a multiplicity of infection (“MOI” hereafter) of 30, in 300 μl of complete RPMI medium, at 37° C., under 5% CO₂. Infected dendritic cells were washed after 2 hours.

### EXAMPLE 2

This example describes how the infected dendritic cells were used to stimulate autologous CD8⁺ T lymphocytes.

As noted, supra, CD8⁺ cells had been isolated, and frozen. The day before stimulation experiments, the CD8⁺ cells were thawed and grown overnight in IMDM, supplemented with 10% human serum, AAG and antibiotics, (complete IMDM), together with 5 U/ml of IL-2. These autologous responder CD8⁺ T lymphocytes were then mixed (1.5×10⁵ cells), with infected, dendritic cells (3×10⁴), in U bottomed microwells in 200 μl of complete IMDM, in the presence of IL-6 (1000 U/ml), and IL-12 (10 ng/ml). On days 6 and 13, autologous dendritic cells were thawed, infected with the ALVAC-MAGE-1 construct in the manner referred to supra, and used to stimulate the responder cells, in medium supplemented with IL-2 (10 U/ml), and IL-7 (5 ng/ml). Aliquots of the T cell microcultures were tested for lytic activity on day 21, as described in the example which follows.

### EXAMPLE 3

The CD8⁺ cells stimulated in example 2 were used in this example. As indicated, they were tested for lytic activity on day 21 of the stimulation experiment described in example 2.

The targets of the CD8⁺ cells were autologous, EBV-B cells that had been infected with either a vaccinia-MAGE-1 virus construct, or control vaccinia virus.

An EBV-B transformed B cell line was derived from the blood cells of the hemochromatosis blood donor referred to supra, by culturing isolated B cells with 20% of a supernatant of EBV-transformed, B95-8 cells, available from the American Type Culture Collection (CRL 1612), in the presence of 1 μg/ml of cyclosporin A. The cells were cultured in Iscove's modified Dulbecco medium, supplemented with 10% fetal calf serum, 0.24 mM L-asparagine, 0.55 mM L-arginine, and 1.5 mM L-glutamine, as well as 100 U/ml penicillin and 100 μg/ml streptomycin.

In order to transfect the EBV-B cells, a readily available vaccinia construct was used, together with parental vaccinia virus as control. The vaccinia construct is referred to, alternatively, as “WR-MAGE-A1” or “vaccinia-MAGE-1.” It encodes the entire MAGE-1 protein.

Virus particles were sonicated for 30 seconds before use. Following sonication, infection was performed on 2×10⁶ target EBV-B cells for 2 hours, using an MOI of 20, in 200 μl of complete RPMI medium. Infected cells were washed, labelled with 100 μCi of Na(⁵¹Cr)O₄, and added to the CD8⁺ cells described supra, at an effector:target ratio of 40:1.

T cells which exhibited activity were taken out of the microcultures described supra. The T cells were cloned in U bottomed microplates via limiting dilution, in complete IMDM medium supplemented with IL-2 (50 U/ml), IL-4 (5 U/ml), and IL-7 (5 ng/ml).

The T cells were stimulated weekly, via addition of phytohemagglutinin, or EBV-B cells that had been transduced with a retroviral construct which encoded MAGE-1, “retro-MAGE-1.NGFR”. These transduced cells were irradiated (100 Gy).

The retro-MAGE-1.NGFR construct was used in order to stimulate anti-MAGE-1 CTLs, and to avoid proliferation of CTLs against adenoviral vector components.

The retro-MAGE-1.NGFR construct is derived from LXSN backbone, which is derived from Maloney murine leukemia virus. It encodes full length MAGE-1, under transcription control of the LTR, and truncated form of human low affinity nerve growth factor, driven by SV40 promoter. EBV-B cells were transduced with this retroviral vector by cocultivation with irradiated, Am12 cells which produced the vector, in the presence of polybrene (0.8 mg/ml) for 72 hours. Pure populations of transduced cells were obtained by immunoselection, using anti-LNGFR monoclonal antibodies, and goat-anti-mouse IgG.

The pure culture of EBV-B cells transduced with the retro-MAGE-1.NGFR construct used, together with allogeneic EBV-B cells (1×10⁴ LG2-EBV-B cells per well), as feeder cells.

Following this protocol, a CTL clone was obtained, referred to as 664/G4.7.

### EXAMPLE 4

The CTL clone discussed in example 3 was tested for its ability to lyse EBV-B cells that had been transfected with constructs which encode MAGE-1.

Vaccinia virus transfectants, as described supra, were tested in a ⁵¹Cr release assay, also as described, supra. Varying effector:target ratios were used, ranging from 30:1 to 0.03:1. Cells were labelled with ⁵¹Cr for one hour, and then combined with the T cells, as noted. Chromium release was measured after 4 hours.

The results indicated that the target cells were lysed.

### EXAMPLE 5

Following the proof that the T cells described herein did lyse cells which expressed MAGE-1, the next step was to determine the MHC molecule which presented the peptide derived from MAGE-1.

The blood donor who provided the cells used in the prior experiments had been typed, previously, as HLA-A3, B7, B37, Cw*0602, Cw7. In order to determine the presenting molecule, 293-EBNA cells were used, and distributed in flat bottomed microwells, one day before transfection with MAGE-1 cDNA inserted in pcDSR-α, as described by Takebe, et al., Mol. Cell Biol 8:466-472 (1988), incorporated by reference. The 293-EBNA cells were also cotransfected with cDNA for one of the HLA molecules discussed supra. In the case of the HLA-Cw7 clone (Cw0704), Cw0602, and A3, the cDNA was inserted in pcDNA3. Also, Cw0701 cDNA was inserted in pEBOSpuro, while B3701, and B7 were inserted in pcDNAIAmp.

Transfections were carried out in microwells, using 50,000 293-EBNA cells, and 50 ng of cDNA for each of the HLA molecules being tested, and MAGE-1, in the presence of 1 μl lipofectamine. One day after transfection, 5000 CTLs were added, in a total volume of 100 μl of complete IMDM, supplemented with 25 U/ml of IL-2.

A TNF release assay was carried out, in accordance with Traversari, et al., J. Exp. Med. 176:1453-1457 (1992), incorporated by reference, with the exception that 20 mM of LiCl was added during incubation of the supernatants with TNF sensitive WEHI 164c13 cells.

The only cells which stimulated the 664/64.7 line were those transfected with both MAGE-A1 and Cw*0602, thus establishing HLA-Cw*0602 as the presenting molecule.

### EXAMPLE 6

This example describes experiments designed to determine the presented peptide.

A set of 16 amino acid long, MAGE-1 derived peptides were prepared, using standard solid phase methodologies.

The peptides, which overlapped by 12 residues and covered the entire MAGE-1 amino acid sequence, were incubated with autologous EBV-B cells that had been labelled with Na(⁵¹Cr)O₄. The cells were incubated for 15 minutes in the presence of 1 μg/ml of peptide, and then tested for lysis with the T cells, at effector:target ratios of 10:1. Chromium release was measured after 4 hours.

The peptides:

| | |
|---|---|
| DGREHSAYGEPRKLLT | (SEQ ID NO:1) |
| and | |
| HSAYGEPRKLLTQDLV | (SEQ ID NO:2) |

were positive. These correspond to amino acids 225-240 and 229-244 of MAGE-1.

The overlap between the peptides, i.e.:

SAYGEPRKL

suggested that this, shorter peptide be tested. It corresponds to amino acids 230-238 of MAGE-1. The shorter peptide was tested, in the same manner as the longer ones.

Half maximal lysis was obtained at a peptide concentration of about 6 nM, which is within the range of previously identified, MAGE derived antigenic peptides. The observed values range from 0.05 to 25 nM. See Chaux, et al., J. Immunol 163:2928-2936 (1999); Luiten, et al., Tissue Antigens 55:149-152 (2000); Schultz, et al., Tissue Antigens 57:103-109 (2001); Traversari, et al., J. Exp. Med. 176:1453-1457 (1992); van der Bruggen, et al., Eur. J. Immunol 24:2134-2140 (1994).

### EXAMPLE 7

Dendritic cells expressing MAGE-1 had been used to activate CTL clone 664/64.7. Hence, experiments were designed to verify that tumor cells process the MAGE-1 molecule to the antigen.

A MAGE-1 expressing tumor cell line, MZ2-MEL.43 was obtained from a patient who tested positive for HLA-Cw6 expression. These cells either were, or were not contacted with gamma interferon (100 U/ml), 48 hours before lysis studies. EBV-B cells were also used. These cells naturally produce TNF. They were fixed with PFA (1% in PBS) for 10 minutes at room temperature, washed twice with Hank's medium, and either pulsed or not with the shorter peptide.

Stimulator cells (2×10⁴) were distributed in flat bottom microwells, and cocultured with 5,000 CTLs, in a total volume of 150 μl of complete IMDM, supplemented with 25 U/ml of IL-2.

TNF production was measured, following overnight co-culture, by testing for toxicity of supernatant on TNF sensitive, WEHI 164 clone 13 cells.

The MAGE-1 expressing tumor line from the HLA-Cw6 positive patient exhibited high production of the TNF by the CTL, either with or without gamma interferon. The EBV-B cells did stimulate TNF production when the peptide was used.

The peptide in question, i.e., SAYGEPRKL, has previously been identified as a CTL epitope for HLA-Cw3 and HLA-Cw16. See Chaux, et al., supra; van der Bruggen, et al., supra. These data show that the molecule can now be extended to HLA-Cw6.

The foregoing disclosure sets forth various features of the invention. These include isolated peptides which are processed to peptides that form immunogenic complexes with HLA-C26 molecules. The peptides of the invention comprise amino acids 6-14 of SEQ ID NO: 1, i.e.:

SAYGEPRKL

concatenated to from 1 to 20 additional amino acids at the N (Ser) or C (Leu) terminus, preferably from 5-10 additional amino acids, such as the peptides of SEQ ID NOS: 1 and 2. Preferably, the concatenated amino acids are identical to the amino acid sequence which precedes Ser or follows Leu in the full length amino acid sequence of MAGE-1, but the concatenated amino acids also accommodate variations, such as conservative substitutions, deletions, additions and so forth. The peptides of the invention possess the functional properties of being taken up by antigen presenting cells, such as dendritic cells, and being processed to the 10 amino acid sequence described supra.

Preferably, the cells which take up the peptides are cells which present HLA-Cw6 molecules on their surface, which makes the peptide more useful than previously, as it can be used in connection with HLA-Cw6 molecules, as well as those which present HLA-Cw3, or HLA-Cw16. As was noted, supra, the 9 amino acid peptide referred to herein is also presented by HLA-Cw3 and Cw16 positive cells, so the cells may also be those which are positive for both HLA-Cw6 and at least one of HLA-Cw3 and HLA-Cw16.

Also a feature of this invention are isolated cytolytic T cells which are specific for complexes of HLA-Cw6 molecules and the 9 amino acid sequence referred to supra, which do not recognize other complexes, including complexes of the sequence and different HLA molecules. As was shown, supra, such cytolytic T cells can be prepared using standard methodologies, including those described herein.

In connection with the cytolytic T cell lines of the invention, various methods can be used to identify and to secure these. Such methodologies include, e.g., FACS or other analytical methods, preferably in combination with molecules, such as tetrameric compounds of avidin or streptavidin, biotin, and HLA/peptide complexes, to identify relevant CTLs from samples.

The ability of the peptides to form recognizable complexes makes them useful as therapeutic agents in conditions such as cancer, such as melanoma where the peptide forms a complex with the HLA molecule, leading to recognition by a CTL, and lysis thereby. As was shown, supra, CTLs which recognize the complexes occur naturally in patients, so administration of the peptide of the invention to an HLA-Cw6 positive subject in need of a cytolytic T cell response is another feature of the invention. Such subjects may be, e.g., cancer patients, such as melanoma patients. Such patients may receive the peptide of the invention, or “cocktails” which comprise more than one peptide, as long as the peptide cocktail includes the peptide of the invention. The peptide component of such cocktails may consist of the peptides described herein, or may combine some peptides disclosed herein with other peptides known in the art, such as the following, which bind to Class I or Class II MHC.

!? ? ? SEQ? ? !? ? ? ID? !PEPTIDE SEQUENCE? ANTIGEN? HLA? NO:

| | | | |
|---|---|---|---|
| YMDGTMSQV | TYROSINASE | A2 | 3 |
| MLLAVLYCL | TYROSINASE | A2 | 4 |
| ELAGIGILTY | MELAN-A | A2 | 5 |
| IMPKAGLLI | MAGE-A3 | A2 | 6 |
| FLWGPRALV | MAGE-A3 | A2 | 7 |
| VRIGHLYIL | MAGE-A6 | Cw7 | 8 |
| YLQLVFGIEV | MAGE-A2 | A2 | 9 |
| FLWGPRALV | MAGE-A12 | A2 | 10 |
| VLPDVFIRC(V) | GnTV | A2 | 11 |
| KASPKIFYV | SSX2 | A2 | 12 |
| GLYDGMEHL | MAGE-A10 | A2 | 13 |
| EVDPIGHLY | MAGE-A3 | A1 | 14 |
| SLLMWITQC | NY-ESO-1 | A2 | 15 |
| IMPKAGLLI | MAGE-A3 | A24 | 16 |
| EVDPIGHLY | MAGE-A3 | B35 | 17 |
| GVYDGREHTV | MAGE-A4 | A2 | 18 |
| EADPTGHSY | MAGE-A1 | A1,B35 | 19 |
| SEIWRDIDF | TYROSINASE | B44 | 20 |
| LPSSADVEF | TYROSINASE | B35 | 21 |
| MEVKPIGHLY | MAGE-A3 | B18,B44 | 22 |
| YRPRPRRY | GAGE-1,2,8 | Cw6 | 23 |
| LAMPFATPM | NY-ESO-1 | Cw3 | 24 |
| ARGPESRLL | NY-ESO-1 | Cw6 | 25 |
| YYWPRPRRY | GAGE-3,4,5,6,7 | A29 | 26 |
| AARAVFLAL | BAGE-1 | Cw16 | 27 |
| TQHFVQENYLEY | MAGE-A3 | DP4 | 28 |
| SLLMWITQCFL | NY-ESO-1 | DP4 | 29 |
| AELVHFLLLKYRAR | MAGE-A3 | DR13 | 30 |
| LLKYRAREPVTKAE | MAGE-A3,A6,A2 | DR13 | 31 |
| AELVHFLLLKYRAR | MAGE-A-12 | DR13 | 32 |
| PEPTIDE SEQUENCE | ANTIGEN | HLA | SEQ ID NO: |
| EYVIKVSARVRF | MAGE-A1 | DR15 | 33 |
| LLKYRAREPVTKAE | MAGE-A1 | DR13 | 34 |
| PGVLLKEFTVSGNILTIRLT | NY-ESO-1 | DR4 | 35 |
| AADHRQLQLSISSCLQQL | NY-ESO-1 | DR4 | 36 |

In an especially preferred embodiment, one administers a cocktail of peptides based upon the HLA profile of the subject being treated. Based upon known Class I peptide binding motifs, such as those set forth by Ramensee, et al., supra, peptides such as those set forth at SEQ ID NOS: 3-36 would be expected to bind to other HLA-Class I or II alleles, such as HLA-A1, A3, B7, B8, B15, B27, B44, B51 in addition to HLA-A2, and subtypes thereof. Further, if appropriate, one or more peptides which bind to HLA-A2, HLA-B7, HLA-B18, B44 and so forth, can be admixed, preferably in the presence of an adjuvant like GM-CSF, alum, or another adjuvants well known to the art, such as CpG. See U.S. Pat. Nos. 6,339,068; 6,239,116; 6,207,646 and 6,194,388, all of which are incorporated by reference. Such combinations of peptides, in the form of compositions, are another feature of the invention, either alone or in combination with such adjuvants. Similarly, one can administer cytolytic T cells specific for the peptide/HLA-Cw6 complexes, such as autologous CTLs, which can be prepared as described in the preceding examples. These CTLs, which are specific for complexes of the 9 amino acid molecules described supra and HLA-Cw6, and no other complexes, are a further feature of the invention.

Yet a further feature of the invention are nucleic acid molecules which consist of nucleotide sequences that encode the peptide of the invention. Such nucleic acid molecules may be used to encode the peptides of the invention, and may be combined into expression vectors, operably lined to a promoter. More than one sequence can be combined in such expression vectors, as can nucleic acid molecules which encode HLA-Cw6 molecules. The constructs can be used to transfect cells, so as to generate the CTLs, or for administration to subjects in need of a cytolytic T cell response or augmenting of a pre-existing T cell response. Such administration could be one of, e.g., administering vector constructs as described, heterologous expression vectors, peptides or recombinant proteins, such as the full length proteins, preferably in recombinant form, from which one or more of the peptides are derived as discussed supra.

The invention also relates to the use of the peptides, CTLs, and other, immunologically active components, such as antibodies, to diagnose pathological conditions such as cancer, melanoma in particular. As was shown, supra, MAGE-1 is expressed in cancer cells and the presence of complexes of the 9 mer and HLA-Cw6 is indicative of a pathological condition. By determining the interaction of the immunologically active component and the complex (by way of, e.g., antibody binding, TNF release, cell lysis, etc.), one can diagnose the pathology, or even determine the status of the pathology via comparing a value to a pre-existing value for the same parameter.

The invention also embraces functional variants of the MAGE-1 HLA class I binding peptide. As used herein, a “functional variant” or “variant” of a MAGE-1 HLA class I binding peptide is a peptide which contains one or more modifications to the primary amino acid sequence of MAGE-1 HLA class I binding peptide and retains the HLA class I and T cell receptor binding properties disclosed herein. Modifications which create a MAGE-1 HLA class I binding peptide functional variant can be made for example 1) to enhance a property of a MAGE-1 HLA class I binding peptide, such as peptide stability in an expression system or the stability of protein-protein binding such as HLA-peptide binding; 2) to provide a novel activity or property to a MAGE-1 HLA class I binding peptide, such as addition of an antigenic epitope or addition of a detectable moiety; or 3) to provide a different amino acid sequence that produces the same or similar T cell stimulatory properties. Modifications to a MAGE-1 HLA class I binding peptide can be made a nucleic acid which encodes the peptide, and can include deletions, point mutations, truncations, amino acid substitutions and additions of amino acids. Alternatively, modifications can be made directly to the polypeptide, such as by cleavage, addition of a linker molecule, addition of a detectable moiety, such as biotin, addition of a fatty acid, substitution of one amino acid for another and the like. Modifications also embrace fusion proteins comprising all of part of the MAGE-1 HLA class I binding peptide amino acid sequence.

The amino acid sequence of MAGE-1 HLA class I binding peptides may be of natural or non-natural origin, that is, they may comprise a natural MAGE-1 HLA class I binding peptide molecule or may comprise a modified sequence as long as the amino acid sequence retains the ability to stimulate T cells when presented and retains the property of binding to an HLA class I molecule such as an HLA-Cw6, Cw3 or Cw16 molecule. For example, MAGE-3 HLA class I binding peptides in this context may be fusion proteins of a MAGE-1 HLA class I binding peptide and unrelated amino acid sequences, a synthetic peptide of amino acid sequences shown in SEQ ID NO: 1 or the sequence SAYGEPRKL labeled peptides, peptides isolated from patients with a MAGE-1 expressing cancer, peptides isolated from cultured cells which express MAGE-1, peptides coupled to nonpeptide molecules (for example in certain drug delivery systems) and other molecules which include the amino acid sequence of SAYGEPRKL.

Preferably, MAGE-1 HLA class I binding peptides are non-hydrolyzable. To provide such peptides, one may select MAGE-1 HLA class I binding peptides from a library of non-hydrolyzable peptides, such as peptides containing one or more D-amino acids or peptides containing one or more non-hydrolyzable peptide bonds linking amino acids. Many non-hydrolyzable peptide bonds are known in the art, along with procedures for synthesis of peptides containing such bonds. Non-hydrolyzable bonds include -psi[Ch.sub.2 NH]-reduced amide peptide bonds, -psi[COCH.sub.2]-ketomethylene peptide bonds, -psi[CH(CN)NH]-(cyanomethlylene) amino peptide bonds, -psi[CH.sub.2CH(OH)]-hydroxyethylene peptide bonds, -psi[CH.sub.2 O]-peptide bonds, and -psi[CH.sub.2 S]-thiomethylene peptide bonds. Methods for determining such functional variants are provided in U.S. Pat. No. 6,087,441, incorporated by reference.

If a variant involves a change to an amino acid of SEQ ID NO: 1 or the sequence SAYGEPRKL, functional variants of the MAGE-1 HLA class I binding peptide having conservative amino acid substitutions typically will be preferred, i.e., substitutions which retain a property of the original amino acid such as charge, hydrophobicity, conformation, etc. Examples of conservative substitutions of amino acids include substitutions made amongst amino acids within the following groups: (a) M, I, L, V; (b) F, Y, W: (c) K, R, H; (d) A, G; (e) S, T: (f) Q, N; and (g) E, D. Methods for identifying functional variants of the MAGE-1 HLA class I binding peptides are provided in a U.S. Pat. Nos. 6,277,956 and 6,326,200 and published PCT application WO0136453 (U.S. patent application Ser. Nos. 09/440,621, 09/514,036, 09/676,005), all of which are incorporated by reference.

Thus methods for identifying functional variants of a MAGE-1 HLA class I binding peptide are provided. In general, the methods include selecting a MAGE-1 HLA class I binding peptide, an HLA class I binding molecule which binds the MAGE-1 HLA class I binding peptide, and a T cell which is stimulated by the MAGE-1 HLA class I binding peptide presented by the HLA class I binding molecule. In preferred embodiments, the MAGE-1 HLA class I binding peptide comprises the amino acid sequence of amino acids 6-14 of SEQ ID NO: 1. More preferably, the peptide consists of the amino acid sequence of SAYGEPRKL. A first amino acid residue of the MAGE-1 HLA class I binding peptide is mutated to prepare a variant peptide. Any method for preparing variant peptides can be employed, such as synthesis of the variant peptide, recombinantly producing the variant peptide using a mutated nucleic acid molecule, and the like.

The binding of the variant peptide to HLA class I binding molecule and stimulation of the T cell are then determined according to standard procedures wherein binding of the variant peptide to the HLA class I binding molecule and stimulation of the T cell by the variant peptide presented by the HLA class I binding molecule indicates that the variant peptide is a functional variant. For example, the variant peptide can be contacted with an antigen presenting cell which contains the HLA class I molecule which binds the MAGE-1 peptide to form a complex of the variant peptide and antigen presenting cell. This complex can then be contacted with a T cell which recognizes the epitope formed by the MAGE-1 HLA class I binding peptide and the HLA class I binding molecule. T cells can be obtained from a patient having a condition characterized by expression of MAGE-1. Recognition of variant peptides by the T cells can be determined by measuring an indicator of T cell stimulation.

Binding of the variant peptide to the HLA class I binding molecule and stimulation of the T cell by the epitope presented by the complex of variant peptide and HLA class I binding molecule indicates that the variant peptide is a functional variant. The methods also can include the step of comparing the stimulation of the T cell by the epitope formed by the MAGE-1 HLA class I binding peptide and the HLA class I molecule, stimulation of the T cell as a determination of the effectiveness of the stimulation of the T cell by the epitope. By comparing the epitope involving the epitope formed by the functional variant with the MAGE-1 HLA class I binding peptide, peptides with increased T cell stimulatory properties can be prepared.

Variants of the MAGE-1 HLA class I binding peptides prepared by any of the foregoing methods can be sequenced, if necessary, to determine the amino acid sequence and thus deduce the nucleotide sequence which encodes such variants.

Other features of the invention will be clear to the skilled artisan, and need not be reiterated herein.

We claim:

1. A method for treating a subject with a pathological condition whose cells characteristic of said pathological condition present HLA-Cw6 molecules on their surface, comprising administering to said subject an amount of a peptide, the amino acid sequence of which consists of the amino acids 6-14 of SEQ ID NO: 1, in an amount sufficient to generate a therapeutically effective, immunologically active responsive against said cells.

2. The method of claim 1, comprising administering said peptide in combination with at least one additional peptide which forms a complex with an MHC molecule other than HLA-Cw6.

3. The method of claim 1, comprising administering said peptide in combination with an adjuvant.

4. The method of claim 1, wherein said immunologically active response is a cytolytic T cell response which causes lysis of cells presenting complexes of said peptide and HLA-Cw6 molecules on their sufaces.

5. The method of claim 1, wherein said pathological condition is cancer.

6. The method of claim 5, wherein said cancer is melanoma.

7. A method for treating a subject with a pathological condition, wherein cells characteristic of said pathological condition present complexes of HLA-Cw6 molecules and a peptide consisting of the amino acids 6-14 of SEQ ID NO: 1 on their surface, comprising administering to said subject an amount of cytolytic T lymphocytes specific for said complexes sufficient to lyse cells presenting said complexes.

8. The method of claim 7, wherein said cytolytic T cells are autologous T cells.

9. The method of claim 7, wherein said pathological condition is cancer.

10. The method of claim 9, wherein said cancer is melanoma.

11. An isolated cytolytic T lymphocyte which recognizes complexes of HLA-Cw6 molecules and the peptide consisting of the amino acids 6-14 of SEQ ID NO: 1, wherein said isolated cytolytic T lymphocyte does not recognize any other complexes of MHC molecules and peptides.

12. A method for determining if a subject is suffering from a pathological condition, comprising administering a sample of cells taken from said subject matter with an immunologically active agent which recognizes complexes of an HLA-Cw6 molecule and the amino acids 6-14 of SEQ ID NO: 1, and determining interaction between said immunologically active agent and said complexes as a determination of said pathological condition.

13. The method of claim 12, wherein said immunologically active agent is a cytolytic T lymphocyte.

14. The method of claim 13, comprising determining lysis of cells by said cytolytic T lymphocyte.

15. The method of claim 13, comprising measuring tumor necrosis factor release by said cytolytic T lymphocyte.

16. The method of claim 12, wherein said immunologically active agent is an antibody.

17. An isolated peptide consisting of from 11 to 30 amino acids, wherein the amino acid sequence of said peptide consists of
SAYGEPRKL
concatenated to from 1 to 20 additional amino acids at Ser or Leu, wherein said peptide is processed by an antigen presenting cell to a peptide consisting of
SAYGEPRKL

18. The isolated peptide of claim 17, consisting of an amino acid sequence found in the amino acid sequence of MAGE-1.

19. The isolated peptide of claim 18, consisting of the amino acid sequence set forth in SEQ ID NO: 1 OR SEQ ID NO: 2.


SEQUENCE LISTING (36 sequences)

SEQ ID NO: 1 - protein, 16 aa, H. sapiens
DGREHSAYGEPRKLLT

SEQ ID NO: 2 - protein, 16 aa, H. sapiens
HSAYGEPRKLLTQDLV

SEQ ID NO: 3 - protein, 9 aa, H. sapiens
YMDGTMSQV

SEQ ID NO: 4 - protein, 9 aa, H. sapiens
MLLAVLYCL

SEQ ID NO: 5 - protein, 10 aa, H. sapiens
ELAGIGILTV

SEQ ID NO: 6 - protein, 9 aa, H. sapiens
IMPKAGLLI

SEQ ID NO: 7 - protein, 9 aa, H. sapiens
FLWGPRALV

SEQ ID NO: 8 - protein, 9 aa, H. sapiens
VRIGHLYIL

SEQ ID NO: 9 - protein, 10 aa, H. sapiens
YLQLVFGIEV

SEQ ID NO: 10 - protein, 9 aa, H. sapiens
FLWGPRALV

SEQ ID NO: 11 - protein, 10 aa, H. sapiens
VLPDVFIRCV

SEQ ID NO: 12 - protein, 9 aa, H. sapiens
KASPKIFYV

SEQ ID NO: 13 - protein, 9 aa, H. sapiens
GLYDGMEHL

SEQ ID NO: 14 - protein, 9 aa, H. sapiens
EVDPIGHLY

SEQ ID NO: 15 - protein, 9 aa, H. sapiens
SLLMWITQC

SEQ ID NO: 16 - protein, 9 aa, H. sapiens
IMPKAGLLI

SEQ ID NO: 17 - protein, 9 aa, H. sapiens
EVDPIGHLY

SEQ ID NO: 18 - protein, 10 aa, H. sapiens
GVYDGREHTV

SEQ ID NO: 19 - protein, 9 aa, H. sapiens
EADPTGHSY

SEQ ID NO: 20 - protein, 9 aa, H. sapiens
SEIWRDIDF

SEQ ID NO: 21 - protein, 9 aa, H. sapiens
LPSSADVEF

SEQ ID NO: 22 - protein, 10 aa, H. sapiens
MEVKPIGHLY

SEQ ID NO: 23 - protein, 8 aa, H. sapiens
YRPRPRRY

SEQ ID NO: 24 - protein, 9 aa, H. sapiens
LAMPFATPM

SEQ ID NO: 25 - protein, 9 aa, H. sapiens
ARGPESRLL

SEQ ID NO: 26 - protein, 9 aa, H. sapiens
YYWPRPRRY

SEQ ID NO: 27 - protein, 9 aa, H. sapiens
AARAVFLAL

SEQ ID NO: 28 - protein, 12 aa, H. sapiens
TQHFVQENYLEY

SEQ ID NO: 29 - protein, 11 aa, H. sapiens
SLLMWITQCFL

SEQ ID NO: 30 - protein, 14 aa, H. sapiens
AELVHFLLLKYRAR

SEQ ID NO: 31 - protein, 14 aa, H. sapiens
LLKYRAREPVTKAE

SEQ ID NO: 32 - protein, 14 aa, H. sapiens
AELVHFLLLKYRAR

SEQ ID NO: 33 - protein, 12 aa, H. sapiens
EYVIKVSARVRF

SEQ ID NO: 34 - protein, 14 aa, H. sapiens
LLKYRAREPVTKAE

SEQ ID NO: 35 - protein, 20 aa, H. sapiens
PGVLLKEFTVSGNILTIRLT

SEQ ID NO: 36 - protein, 18 aa, H. sapiens
AADHRQLQLSISSCLQQL